(19) United States
(12) Patent Application Publication
(10) Pub. No.: US 2026/0354764 A1
(43) Pub. Date: Nov. 12, 2026

(54) INTRAVASCULAR RESISTANCE MEASUREMENT DEVICE AND RELATED METHOD FOR INTERPRETING VASCULAR CONDITIONS

(71) Applicant: ProMedica Health Systems, Inc., Toledo, OH (US)

(72) Inventors: Fedor Lurie, Toledo, OH (US); Todd Russell, Toledo, OH (US)

(21) Appl. No.: 19/745,709

(22) Filed: Jul. 17, 2026

Related U.S. Application Data
(63) Continuation of application No. 18/218,737, filed on Jul. 6, 2023.
(60) Provisional application No. 63/367,784, filed on Jul. 6, 2022.
(60) Provisional application No. 63/457,891, filed on Apr. 7, 2023.

(51) Int. Cl. A61B 5/00; A61B 5/02; A61B 5/027; A61M 25/10

(52) U.S. Cl. CPC A61B 5/6853; A61B 5/02007; A61B 5/027; A61M 25/1011

(57) ABSTRACT

A system and method for interpreting vascular conditions of a blood vessel. The system comprises an intravascular device having a catheter tube, first and second selectively inflatable balloons spaced apart along the catheter tube, and one or more ports axially between the balloons; one or more sensors in fluid communication with the one or more ports and configured to measure at least one of pressure and flow rate of fluids of the vascular system between the balloons; and a controller that receives sensor readings, determines a resistance to flow across a plurality of instances of time, and outputs a quantitative or qualitative indication of a vascular condition of the blood vessel. Adjustable spacing between the balloons, differential balloon compliance for lower-pressure distal occlusion, adjustable first-balloon inflation for clearance variation, and in-vessel therapy with post-therapy re-measurement are also described.

### CROSS-REFERENCE TO RELATED APPLICATIONS

This application is a continuation of US Application Serial No. 18/218,737 filed Jul. 6, 2023, which claims the benefit of US Provisional Application Serial No. 63/367,784 filed Jul. 6, 2022 and US Provisional Application Serial No. 63/457,891 filed Apr. 7, 2023, the disclosures of each of which are hereby incorporated by reference as if fully restated herein.

### TECHNICAL FIELD

Exemplary embodiments relate generally to devices, systems, and methods for measuring fluid resistance within a blood vessel, such as a vein of a human being, for interpreting vascular conditions, such as vascular occlusions or stenosis.

### BACKGROUND AND SUMMARY OF THE INVENTION

Sometimes, blood vessels become fully or partially occluded and/or stenosed, such as from one or more lesions. This can cause issues with adequate circulation, resulting in various medical complications. Various techniques are known for treating such occluded and/or stenosed vessels, such as to improve blood flow. Often, it is useful to know the extent of such occlusion, such as length, amount, and/or type of occlusion. However, it is difficult to measure such intravascular features in a living patient, particularly in a relatively non-invasive manner. Known techniques for investigating such occlusions include various imaging techniques such as, for example, intravascular ultrasound (IVUS), CT, MRI, and venography which is a catheter-based diagnostic procedure used to image the inside of a vein, providing essentially a real-time view. These techniques are limited in that they rely on appearance. Generally, veins and other blood vessels are relatively compliant, which makes an accurate assessment based on appearance difficult. Regardless, known imaging techniques are generally limited to only one focal plane at a time, among other drawbacks. Furthermore, such measurements generally require inflating blood vessels to artificially high pressures which fail to accurately mimic biological vessel conditions.

What is needed is alternative means for measuring characteristics of an interior of a blood vessel. Devices, systems, and methods for measuring resistance characteristics within a vascular system, such as a vein of a human being are provided, which may permit the assignment of hemodynamic significance to the abnormality or lesion. In exemplary embodiments, the resistance characteristics may be interpreted to derive an understanding of vascular conditions, such as but not limited to, blockages or stenosis.

An intravascular device may comprise at least two balloons at a distal end which are spaced apart from one another. The distal end of the device may be inserted into the vascular system of a person undergoing study and negotiated, such as over a guidewire, to a site of study, which may include one or more blockages though such is not necessarily required. The area between the two balloons may define an area of study. A first, proximal one of the balloons may be inflated to fully or substantially occlude normal flow of the blood vessel. A fluid may be introduced, such as through ports along a catheter tube between the two balloons in a controlled flow or pressure. A second, distal one of the balloons may be adjustably inflated to induce changes to characteristics of the induced flow. In this fashion, the volume and/or pressure of the flow may be known such that resistance forces may be derived.

The induced flow may be altered, such as by pressure of induced flow (e.g., by adjusting fluid ejected and/or adjusting inflation of a distal balloon), volume of area under study (e.g., by changing relative position of the two balloons, thereby adjusting a length of the area of study), inflating, deflating, or repositioning the balloons (e.g., adjusting clearance relative to an occlusion). Resulting changes to flow rates and/or resistance forces may be measured.

More elastic vessels may provide relatively consistent resistance forces to changes in volume or pressure as the vessel may volumetrically adjust to accommodate changes in the flow. Less elastic vessels may provide relatively dynamic resistance forces to changes in volume or pressure as the vessel may be unable to volumetrically adjust. The elasticity of the vessel may be an indicator of vessel health, with relatively inelastic vessels indicating a relatively stenosed or diseased vessel and relatively elastic vessels indicating relatively healthy vessels.

Alternatively, or additionally, as clearance between the distal balloon(s) and the surrounding tissue increase (e.g., looser fit), flow rate past the distal balloon(s) may increase and/or backpressure may decrease. As clearance between the distal balloon(s) and the surrounding tissue decreases (e.g., tighter fit), flow rate past the distal balloon(s) may decrease and/or backpressure may increase. In this fashion, the size of the vessels and/or blockages may be measured.

The balloon(s) may be used subsequently to perform therapies, such as but not limited to, angioplasty, stent placement, drug delivery (e.g., by drug coating), combinations thereof, or the like.

Measurements may be performed at one or more areas, such as to generate data, interpretations, and/or visualizations of blood vessels, such as veins of the human vascular system. Measurements may be taken before and/or after treatment, as another example, to assess the impact of treatments.

The distal balloon(s) and/or occluding balloon(s) may optionally be used to perform various therapies at the blood vessel, such as angioplasty.

While veins are sometimes discussed herein, these disclosures may be utilized for arterial or other vasculature applications, by way of further non-limiting example.

Further features and advantages of the systems and methods disclosed herein, as well as the structure and operation of various aspects of the present disclosure, are described in detail below with reference to the accompanying figures.

### BRIEF DESCRIPTION OF THE DRAWINGS

In addition to the features mentioned above, other aspects of the present invention will be readily apparent from the following descriptions of the drawings and exemplary embodiments, wherein like reference numerals across the several views refer to identical or equivalent features, and wherein:

FIG. 1 is plan view of an exemplary intravascular measurement system also indicating section line A-A and detail A;

FIG. 2 is a detailed side view of a distal end of a vascular measurement device of the system of FIG. 1;

FIG. 3 is a detailed side view of another exemplary embodiment of the distal end of the vascular measurement device of the system of FIG. 1;

FIG. 4 is a front sectional view taken along section line A-A of FIG. 1 for the embodiments of FIGS. 1-3;

FIG. 5 is a flow chart with exemplary logic for operating the system and devices of FIGS. 1-4;

FIG. 6 is a detailed side sectional view of a distal end of another exemplary embodiment of the vascular measurement device of the system of FIG. 1, taken along section line B-B of FIG. 7;

FIG. 7 is a front sectional view taken along section line A-A of FIG. 1 for the embodiment of FIG. 6;

FIG. 8 is a flow chart with exemplary logic for operating the system and devices of FIGS. 6-7;

FIG. 9 is a plan view of an exemplary visualization display which may be generated by the systems, devices, and/or methods of FIGS. 1-8;

FIG. 10 is a plan view of the systems, devices, and/or methods of FIGS. 1-9 in use in an exemplary blood vessel;

FIG. 11 is a plan view of the system of FIG. 10 in use with a second balloon more fully inflated; and

FIG. 12 is a plan view of another exemplary embodiment of the system of FIGS. 10-11 in use in another exemplary blood vessel.

### DETAILED DESCRIPTION OF EXEMPLARY EMBODIMENT(S)

Various embodiments of the present invention will now be described in detail with reference to the accompanying drawings. In the following description, specific details such as detailed configuration and components are merely provided to assist the overall understanding of these embodiments of the present invention. Therefore, it should be apparent to those skilled in the art that various changes and modifications of the embodiments described herein can be made without departing from the scope and spirit of the present invention. In addition, descriptions of well-known functions and constructions are omitted for clarity and conciseness.

Embodiments of the invention are described herein with reference to illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

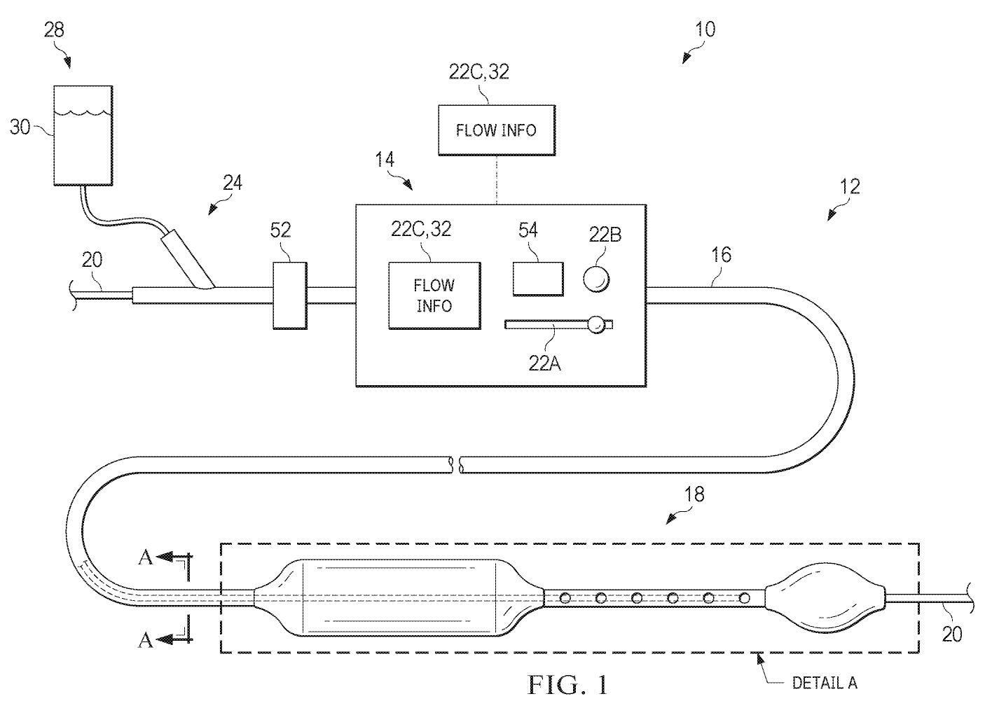

FIG. 1 illustrates an exemplary system 10 for measuring vascular characteristics within a passageway. In exemplary embodiments, without limitation, the system 10 may be configured to measure blood flow resistance within veins of human beings, though the system 10 may be adapted to measure any type of fluid within any type of vascular passageway of any type of animal. Flow rates and/or resistance characteristics measured may be interpreted to, and thus indicative of, certain vascular conditions, such as but not lot limited to presence, size, and/or nature of occlusions and/or level of stenosis.

Relationships between pressure, force, and volume are known. For example, it is known that pressure equals force times volume. Thus, by controlling volume and pressure, force may be determined. In the case of vascular architecture, where a pressure of a known amount may be provided at a known area of study (i.e., volume), a force may be determined. As pressure and/or volume are adjusted, changes to the force, or lack thereof, may be indicative of relatively elastic (typically, healthy) passages or relatively hardened and/or stenosed (typically, diseased) passages.

A flow having certain known characteristics may be provided and/or induced within a portion of the vascular system. In this fashion, a pressure of fluid within an area of study may be known. The flow may be partially, substantially (e.g., ≥95%), or fully (e.g., ≥99%) isolated to an area of study, such as by a proximal, first balloon 42 (sometimes referred to herein as the occluding balloon) and a second balloon 44 (sometimes referred to herein as the variable size, measurement, or therapeutic balloon). In this fashion, the volume of an area under study may be known and/or defined, such as based on the size of the vascular region isolated between the balloons 42, 44. As the pressure and volume of fluid in the area of study may be known, a resistance force to the flow may be determined.

One or more of the volume of the area under study and/or pressure of the flow may be altered, such as by injecting additional fluid (e.g., saline, dye, combinations thereof, or the like), removing some of the fluid, adjusting a flow rate of the fluid injected, and/or movement of the first balloon 42 relative to the second balloon 44. Doing so may result in changes to flow rates and/or resistance forces to be measured under the varying conditions. The tissue response to the changes may indicate a level of vascular elasticity, and thus health or condition. A relatively higher level of elasticity may result in relative constant resistance measurements when changes to induced flow pressure and/or volume are provided, as the vasculature may adjust volumetrically, for example, to accommodate relatively higher pressures and/or increased flow. A relatively lower level of elasticity may result in a relatively dynamic resistance measurements under varying volume and/or pressure of flow as the vasculature may be less able to adjust volumetrically.

Alternatively, or additionally, as clearance between the distal balloon(s) 44 and the surrounding tissue increase (e.g., looser fit), flow rate past the distal balloon(s) 44 may increase and/or backpressure may decrease. As clearance between the distal balloon(s) 44 and the surrounding tissue decreases (e.g., tighter fit), flow rate past the distal balloons 44 may decrease and/or backpressure may increase. In this fashion, the size of the vessels and/or blockages may be measured.

Measurements may be performed across various segments of the blood vessel to gauge blood elasticity and/or condition. Measurements may be provided before and/or after treatment to gauge effect of treatment.

The system 10 may include a catheter device 12. The catheter device 12 may comprise a distal portion 18. The distal portion 18 may be connected to a catheter tube 16. The catheter tube 16 may extend from a handle subassembly 14. The catheter tube 16 and/or the distal portion 18 may be configured for placement within, and/or navigation through, some or all of a human vascular system. For example, without limitation, the catheter tube 16 and/or the distal portion 18 may be flexible, may comprise one or more biocompatible materials, and/or may be configured for one time or regular sterilization and reuse.

The handle subassembly 14 may comprise one or more control mechanisms 22 for the distal portion 18. Such control mechanisms 22 may include, for example without limitation, one or more slides 22A, knobs 22B, levers, switches, buttons, electronic touch displays, combinations thereof, or the like. The control mechanisms 22 may be configured to electronically and/or mechanically control various operations of the distal portion 18, the device 12, and/or the system 10 more generally. For example, without limitation, the control mechanisms 22 may be mechanically linked to elements of the distal portion 18, the device 12, and/or the system 10 more generally, such as by way of wires, sleeves, tubes, rods, linkages, flexible lines, gears, cams, combinations thereof, or the like. Alternatively, or additionally, the control mechanisms 22 may be electronically linked to elements of the distal portion 18, the device 12, and/or the system 10 more generally, such as by way of wired or wireless connection to motors, actuators, combinations thereof, or the like.

In exemplary embodiments, without limitation, a first one of the control mechanisms 22A may be configured to control movement of one or more balloons 42, 44 of the distal portion 18. Such movement control may include inflation/deflation, axial movement towards or away from one another, axial movement relative to the handle subassembly 14, combinations thereof, or the like.

One or more fluid passageways, mechanical linkages, and/or electronic linkages, and/or other components may extend through some or all of the catheter tube 16. In exemplary embodiments, without limitation, the catheter tube 16 may be configured to accommodate a guide wire 20. In this fashion, the distal portion 18 may be delivered along the guide wire 20 to a zone of interest within the vascular system.

The handle subassembly 14 and/or the catheter tube 16 may be configured for fluid connection with one or more fluid sources 28. The fluid sources 28 may comprise one or more containers 30 for one or more types or kinds of fluids, such as but not limited to, saline, dyes, combinations thereof, or the like. Any type or kind of fluid may be utilized. Such connection may be made by way of one or more fluid ports 24.

The system 10 may comprise one or more sensors 52. The sensors 52 may comprise pressure sensors, force sensors, flow rate sensors, combinations thereof, or the like. The sensors 52 may be fluidly interposed between the fluid source(s) 28 and the distal portion 18, which as further explained herein, may be in fluid communication with an ambient environment, such as but not limited to, a vascular environment in which the distal portion 18 is operated. In this fashion, flow rate, resistance forces, pressures, combinations thereof, or the like may be measured for blood vessels when the distal portion 18 is navigated to a zone of interest within a patient’s vascular system. Any number, type, and/or placement of sensors 52 may be utilized. For example, without limitation, the sensors 52 may be placed at the distal portion 18 and in wired or wireless connection with other elements of the system 10 and/or device 12.

The system 10 may comprise one or more controllers 54. The controllers 54 may be local to the device 12, remote therefrom, combinations thereof, or the like. For example, without limitation, some or all of the controllers 54 may be provided at personal electronic devices and/or servers. The controllers 54 may be in electronic communication with the sensors 52, and may be configured to receive and/or interpret data received from the same. The controllers 54 may, alternatively or additionally, be in physical and/or electronic connection with the control mechanisms 22, and may be configured to receive and/or interpret data received from the same. The controllers 54 may be configured to control operations of the device 12 and/or system 10 based on data received from the sensors 52 and/or control mechanisms 22.

In exemplary embodiments, without limitation, the system 10 may comprise one or more displays 32. The displays 32 may be configured to provide information regarding device 12 status, fluid introduction information, measured flow and/or resistance characteristics, combinations thereof, or the like. The displays 32 may operate as displays only and/or interactive user interfaces 22C. When referred to herein as a display 32 or user interface 22C, use as one or both as a display 32 or user interface 22C is intended. Content generated at the displays 32 may be provided by the controllers 52 in exemplary embodiments, without limitation. The displays 32 may be provided at the device 12, such as at the handle subassembly 14, and/or remote therefrom, such as at one or more dedicated displays and/or personal electronic devices.

Information provided at the displays 32 may include data readouts (e.g., pressure readings, flow readings, flow rates, resistance forces, combinations thereof, or the like), vascular condition information (e.g., openings, blockages, clearance, elasticity, stenosis levels, combinations thereof, or the like), historical information, cumulative information, averages, high readings, low readings, most common readings, median readings, bar charts, line charts, color coded results (e.g., red for outside or normal, green for within normal parameters), pass/fail indications, dials, combination thereof, or the like.

The information provided at the displays 32 may be qualitative or quantitative in nature. The device 12 and/or sensors thereof may be calibrated to various parameters, such as based on specific readings (e.g., in mmHg, ml/min, wood, Pa/m/s, combinations thereof, or the like). The handle subassembly 14, other portion of the device 12 or the system 10 may include one or more speakers or other audio generating devices configured to provide audible feedback regarding vascular condition. For example, without limitation, a clicking noise may be provided based on experienced resistance. As measured resistance increases, the pace of the clicking sound may be increased. This may provide the user with a hands-free qualitative type of feedback during use.

Features and operations of the device 12 to measure vascular conditions are further discussed herein.

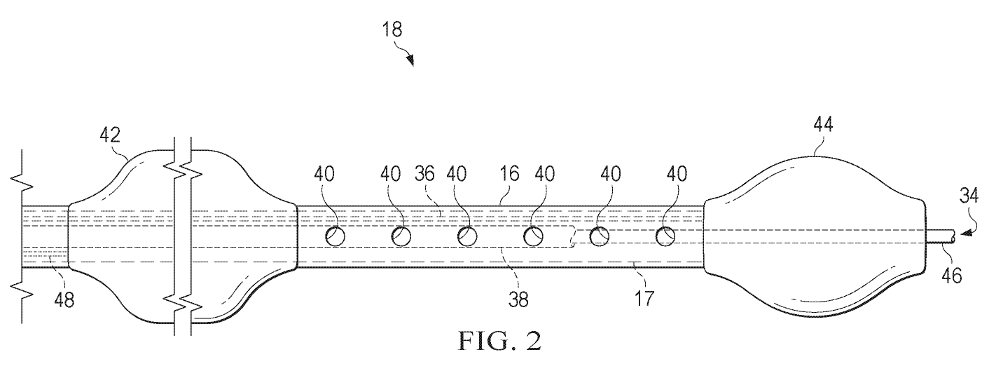

FIG. 2 illustrates an exemplary embodiment of the distal end 18. The distal end 18 may comprise one or more balloons 42, 44. A first balloon 42 and a second balloon 44 may be provided. The first balloon 42 may be proximal to the handle subassembly 14 along the catheter tube 16 relative to the second balloon 44, though such is not required.

The second balloon 44 may be configured to fully or substantially occlude the blood vessel upon adequate inflation (e.g., ≥95% occlusion). The second balloon 44 may be relatively compliant to facilitate such total or substantial occlusion at relatively lower pressures, such as in comparison to the first balloon 42. The second balloon 44 may be 1 cm in length, though any size may be utilized. The second balloon 44 may be inflatable up to 14mm in diameter, though any size may be utilized. The size and/or compliance level of the second balloon 44 utilized may vary based on the patient and/or portion of the vascular system in which the device 12 is utilized.

One or more fluid passageways 36 may extend through some or all of the catheter tube 16 to the second balloon 44 to permit inflation and deflation of the same. The fluid passageway(s) 36 may comprise one or more tubes, by way of non-limiting example.

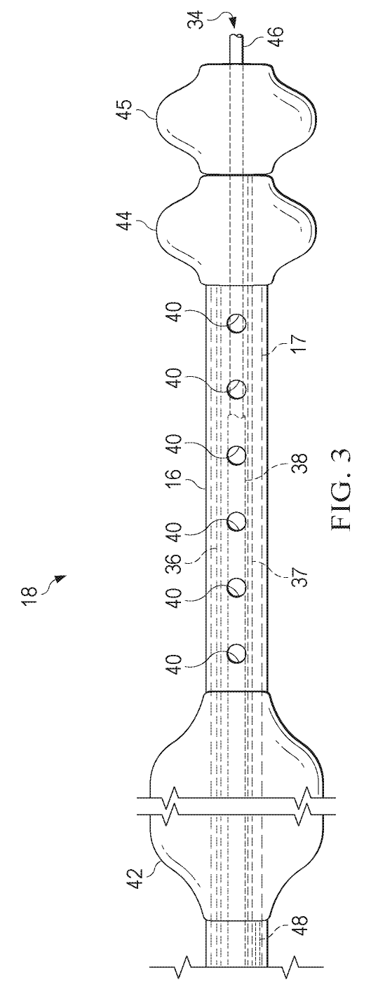

Alternatively, such as illustrated with particular regard to at least FIG. 3, two balloons 44, 45 may be provided to occlude the blood vessel or other vascular region, which may be individually inflatable. The two balloons 44, 45 may be 2cm each in length, though any size may be utilized. Any number of balloons or any size, type, and/or arrangement may be utilized to occlude the vessel. Separate fluid passageways 36, 37 may be provided for each of the balloons 44, 45, such as to provide individual inflation/deflation, though such is not required. In other exemplary embodiments, one or more common fluid passageways may be utilized.

The first balloon 42 may be relatively less compliant, such as compared to the second balloon(s) 44, 45. The first balloon 42 may be adjustably inflated to various diameters, in exemplary embodiments without limitation. In exemplary embodiments, without limitation, the first balloon 42 may be known to inflate to various sizes at various inflation pressures, volumes, external conditions (e.g., ambient fluid pressure), combinations thereof, or the like. One or more fluid passageways 48 may extend to the first balloon 42, such as to permit individual inflation/deflation of the same. In other exemplary embodiments, without limitation, one or more common fluid passageways may be utilized.

One or more fluid introduction passageways 38 may be provided. The fluid introduction passageway 38 may extend through some or all of the catheter tube to a space between the first balloon 42 and the second balloon 44. The fluid introduction passageway 38 may be in fluid communication with a series of one or more ports 40 within the catheter tube 16. In this fashion, fluid from the one or more fluid sources 28 may be selectively introduced into the vessel. This may, alternatively or additionally, facilitate the measurement of fluid pressures within the vessel, such as by way of back pressure. Any number of portion 40 of any size, shape, arrangement, and/or distancing may be utilized. For example, without limitation, multiple such ports 40 may be linearly distributed and/or be circumferentially distributed about the catheter tube 16.

A guidewire passageway 46 may extend through some or all of the catheter tube 16, such as to accommodate a guidewire 20. The guidewire passageway 46 may terminate at a distal opening 34 configured to accommodate the guidewire 20. The guidewire passageway 46 may extend within the fluid introduction passageway 38, such as in a nested arrangement, though such is not necessarily required.

In exemplary embodiments, without limitation, the catheter tube 16 may be configured to accommodate a second catheter tube 17 connected to the second balloon 44. In this fashion, the second balloon 44 may be independently moved and/or controlled. The second catheter tube 17 may accommodate the passageway 46 and/or guidewire 20, and/or one or more of the fluid passageways (e.g., fluid passageway 36), by way of non-limiting example. In this fashion, the second balloon 44 may be extendable through the first balloon 42, though such is not necessarily required.

Measurements may be made relative to anticipated local conditions. For example, without limitation, different vessels may be expected to have different levels of resistance or different changes. Determinations of vessel condition may be made manually or automatically based on absolute thresholds, percent changes, rate changes, combinations thereof, or the like to resistance, which may be varied based on indicated placement of the device 12.

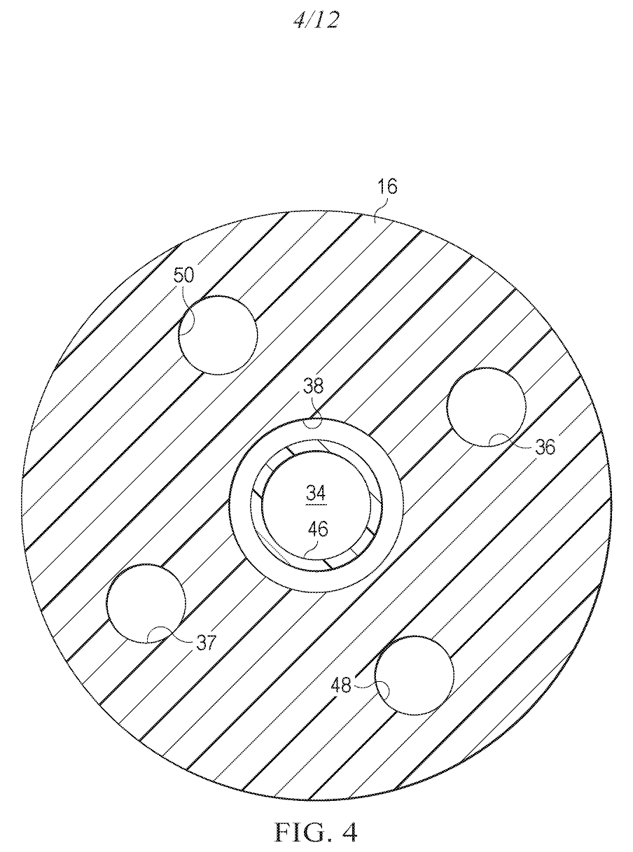

FIG. 4 illustrates an exemplary cross-sectional view of the catheter tube 16. The size, shape, location, number, and/or arrangement of the passageways 36, 37, 38, 46, and/or 48 may be varied. Other mechanical (e.g., wires, rope, cable, members, sleeves, combinations thereof, of the like) and/or electrical linkages (e.g., wires) may be provided within the catheter tube 16, such as part of, or in substitution for one or more of the passageways 36, 37, 38, 46, and/or 48, such as to control movement of one or both of the balloons 42, 44.

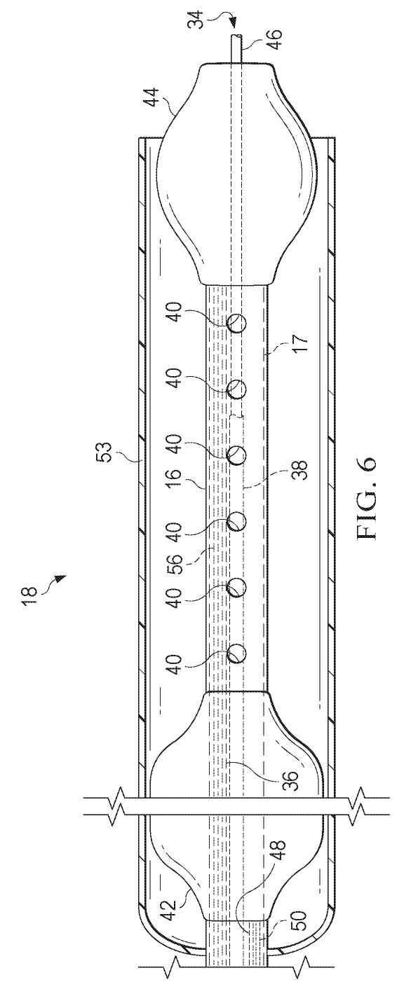

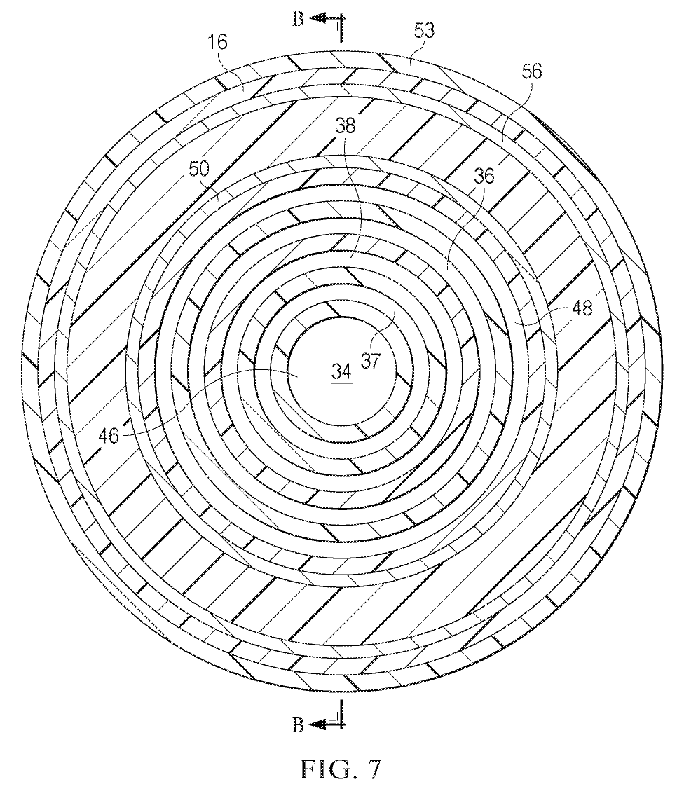

For simplicity of illustration, some or all of the passageways and/or linkages in several of the figures may be illustrated in simplified form. These may include, for example without limitation, passageways 36, 38, 46, and 48 of FIGS. 2-3 and 6 and linkage 40 of FIG. 6. These passageways may be illustrated as a line (solid or broken) so as to indicate their pathway without introducing unnecessary and potentially confusing detail and complexity to the drawings. However, as those of skill in the art will appreciate, such passageways, in practice, may comprise one or more tubes, channels, or the like, which may be separately provided and/or nested within one another, such as illustrated and/or described with regard to at least FIGS. 4 and 7, by way of non-limiting example.

The control mechanisms 22, which may be local to, or external to, the handle subassembly 14 and/or device 12 may include, for example without limitation, valves, pumps, motors, nozzles, combinations thereof, or the like, such as to control the selective introduction or removal of inflationary fluid to the balloon 42, 44, 45. The inflationary fluid may comprise one or more fluids from the fluid sources 28 (e.g., saline), ambient air, combinations thereof, or the like. Such control mechanisms 22 may, alternative or additionally, be used to control selective introduction of fluids into the vessel, such as by way of the port(s) 40.

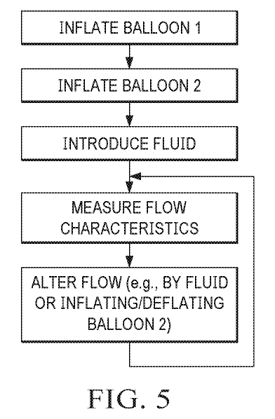

FIG. 5 illustrates an exemplary method for use of the system 10. The first, proximal, occluding balloon 42 and/or distal balloon(s) 44, 45 may be inflated, such as to fully or substantially occlude the vessel (e.g., ≥ 95%). The first balloon 42 and/or variable size balloon(s) 44, 45 may be known to inflate to various diameters with the introduction of particular volumes of inflationary fluid, increase of inflationary fluid up to a predetermined pressure, by material characteristic(s), combinations thereof, or the like. The volume of the area between the balloons 44, 45 and 42 may be known, such as based on distance between the balloons 44, 45 and 42 and/or diameter of the vessel. The distance between the proximal balloon 42 and the distal balloon(s) 44, 45 may be fixed or adjustable. When fixed, the distance may be known. When adjustable, the distance may be electronically recorded by the device 12 and/or manually read by spaced apart radiopaque or other visual indicators spaced apart along the catheter tube 16. In other exemplary embodiments, the volume may be determined, at least in part, based on the size to which one or more of the balloons 42 and 44, 45 are inflated.

One or more fluids may be introduced into the vessel, such as from the fluid sources 28 through the ports 40, to create a flow at a known pressure within the vessel. In exemplary embodiments, without limitation, the pressure of the fluid may be provided at or near to biological pressures, such as but not limited to in the range of about 30-35 mm hg, though any pressure(s) may be utilized. In this fashion, by way of non-limiting example, vascular areas under biological conditions may be studied. The balloon(s) 42, 44, 45 may be selectively inflated/deflated to control the balloon(s) 42, 44, 45 to various sizes (e.g., diameters) and/or pressures.

Resulting flow and/or resistance characteristics may be determined based on the known pressure and/or volume and/or measured, such as by way of the sensors 52.

One or more of the volume of the area under study, pressure of the flow, and/or volume of the flow may be altered, such as by injecting additional fluid (e.g., saline), removing some of the fluid, changing flow rather of the fluid, and/or movement of the first balloon 42 relative to the second balloon(s) 44, 45. Doing so may result in changes to resistance forces to be measured under the varying conditions. The tissue response to the changes may indicate a level of vascular elasticity and thus health or condition. A relatively higher level of elasticity may result in relative constant resistance measurements when changes to induced flow pressure and/or volume are provided, as the vasculature may adjust volumetrically, for example, to accommodate higher pressures and/or increased flow. A relatively lower level of elasticity may result in a relatively dynamic resistance measurements under varying volume and/or pressure of flow as the vasculature may be less able to adjust volumetrically.

Measurements may be performed across various segments of the blood vessel to gauge blood elasticity and/or condition. Measurements may be provided before and/or after treatment to gauge effect of treatment.

The controller(s) 54 may be configured to receive data from the sensors 52 or otherwise inputted (e.g., known, inputted manually) to determine vessel size, blockage characteristics (e.g., percent blockage), vessel characteristics (e.g., level of stenosis), combinations thereof, or the like. The controller(s) 54 may be configured to provide raw results and/or interpretations of the data at the displays 32. Such interpretations may include, by way of non-limiting example, visualizations of the blockage and/or vessel.

Measurements may be taken at a single or at multiple locations, such as along a length of blood vessel to measure a blockage along its entire length.

Veins of the human vasculature may be particularly well suited for such measurements due to their relatively elastic nature, however other vascular passageways may be so studied.

The balloons 42, 44, and/or 45 may, alternatively or additionally, be used to provide a therapeutic effect. For example, without limitation, the balloons 42, 44, and/or 45 may be used to perform angioplasty, stenting, drug delivery (via drug coating and/or the ports 40). In this fashion, the device 12 may be used to diagnose a level of stenosis, occlusion, and/or other condition, perform a therapy (e.g., angioplasty), and take a subsequent reading of the resulting flow and/or resistance and vascular condition, potentially without ever leaving the vessel.

Dyes or other materials may be provided, such as for additional imaging.

FIG. 6 illustrates another exemplary embodiment of the distal end 18 where one or both of the balloons 42, 44 are adjustable, such as in a sliding fashion along a longitudinal axis of the guidewire 20 and/or device 12. In exemplary embodiments, without limitation, a linkage 50, such as but not limited to, a wire, rope, cable, sleeve, combinations thereof, or the like, may be provided which connect the first balloon 42 to one or more of the control devices 22, such as to permit axial movement (e.g., advancement, retraction) of the first balloon 42 axially along the catheter tube 16 towards the second balloon 44 or vice versa (e.g., axial retraction/advancement of the second balloon 44 toward the first balloon 42). In this fashion, fluid pressure may be increased resulting from the decreasing volume. Optionally, such movement may be made within a sheath 53. Alternatively, or additionally, the second balloon 44 and/or first balloon 42 may be axially moved, such as by way of one or more linkages 56.

In exemplary embodiments, without limitation, a relatively consistent fluid flow and/or pressure may be maintained while the balloons 42, 44 are moved relative to one another, such as to examine a relatively large area of study. For example, without limitation, the second balloon 44 may be inflated to a size somewhat smaller than a diameter of the vessel under study. A flow may be introduced, such as through the ports 40 at a controlled pressure. As the second balloon 44 is moved, such as towards the first balloon 42, and an occlusion is encountered, monitored resistance may increase, such as due to the localized narrowing of clearance between the second balloon 44 and the proximate wall of the occlusion. In this fashion, the device 12 may be quickly moved across a large area permitting a relatively large area of study and/or allow less exact placement of the device 12.

FIG. 7 illustrates an exemplary cross section of the catheter tube 16. Any number, size, type, and/or arrangement of the passageways 36, 37, 38, 46, 48, and/or linkages 50, 56 may be utilized. For example, without limitation, some or all such passageways 36, 37, 38, 46, 48, and/or linkages 50, 56 may be nested and/or placed adjacent to one another.

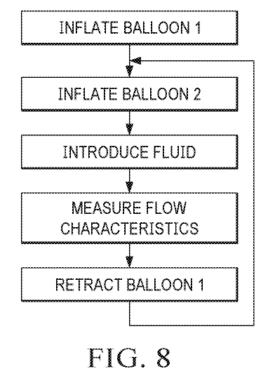

FIG. 8 illustrates an exemplary method for use of the system 10. The first, proximal balloon 42 may be inflated, such as to fully or substantially occlude the vessel (e.g., ≥95%). The second, distal balloon 44 may be selectively inflated/deflated to control the second balloon 44 to various sizes (e.g., diameters) and/or pressures. The first and/or second balloon 42, 44 may be known to inflate to various diameters with the introduction of particular volumes of inflationary fluid, increase of inflationary fluid up to a predetermined pressure, by material characteristic(s), combinations thereof, or the like. The second balloon 44, once inflated to at least a nominal pressure, may be retracted and/or otherwise moved relative to the first balloon 42 to create increased pressure and/or reduced volume. Fluid 28 may be optionally introduced into the vessel, such as by way of the ports 40, though such is not necessarily required.

Resulting flow and/or resistance characteristics may be determined and/or measured, such as by way of the sensors 52. Changes, rate of change, or lack of rate of change to resistance may be used to judge vessel health. In exemplary embodiments, without limitation, as clearance between the second balloon 44 and the surrounding tissue decrease (e.g., tighter fit) the resulting pressure and/or resistance may increase and/or flow may decrease. As the clearance between the second balloon 44 and the surrounding tissue increases (e.g., looser fit) the resulting pressure and/or flow may decrease and/or flow may increase. Performing such measurements along multiple portions of the vasculature at or at various flow conditions (e.g., volumetric flow rate, pressure, balloon 44 size, combinations thereof, or the like) may be used to interpret vessel size and/or condition. For example, without limitation, blockages may be measured as decreasing vessel diameters of various distances. Alternatively, or additionally, heavily stenosed regions may be more inelastic. In this fashion, the tissue response may be measured under changing conditions, such as to determine elasticity levels.

The controller(s) 54 may be configured to receive data from the sensors 52 or otherwise inputted (e.g., known, inputted manually) to determine vessel size, blockage characteristics (e.g., percent blockage), vessel characteristics (e.g., level of stenosis), combinations thereof, or the like. The controller(s) 54 may be configured to provide raw results and/or interpretations of the data at the displays 32. Such interpretations may include, by way of non-limiting example, visualizations of the blockage and/or vessel.

Dyes or other materials may be provided, such as for additional imaging.

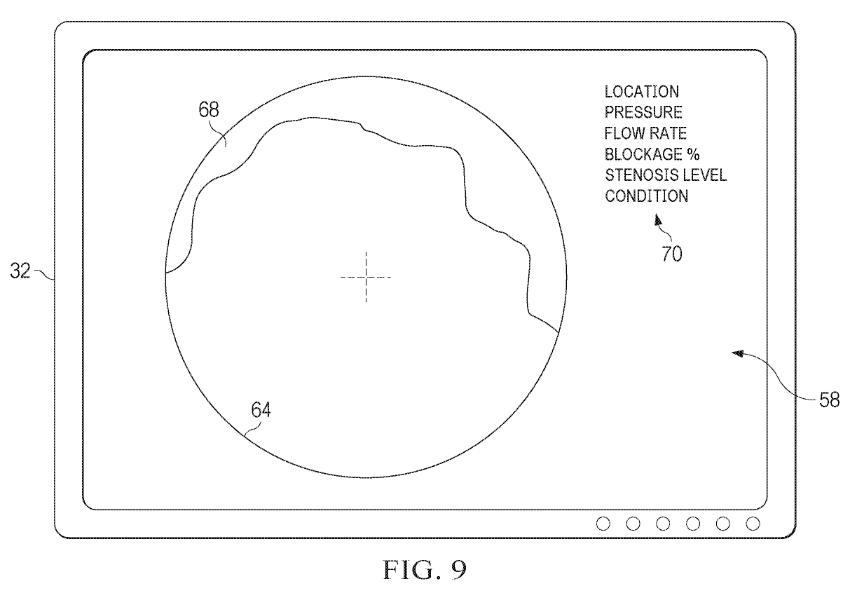

An example of a visualization 58 provided by the system 10 is provided at FIG. 9, without limitation. The blood vessel 64, including blood vessel wall, and/or blockages 68 may be visualized, and various information 70 may be presented.

Measurements may be taken at a single or at multiple locations, such as along a length of a blood vessel to measure a blockage along its entire length.

Veins of the human vasculature may be particularly well suited for such measurements due to their relatively elastic nature, though other vascular passageways may be so studied.

The balloons 42, 44, and/or 45 may, alternatively or additionally, be used to provide a therapeutic effect. For example, without limitation, the balloons 42, 44, and/or 45 may be used to perform angioplasty, stenting, drug delivery (via drug coating and/or the ports 40). In this fashion, the device 12 may be used to diagnose a level of stenosis, occlusion, and/or other condition, perform a therapy (e.g., angioplasty), and take a subsequent reading of the resulting flow and/or resistance and vascular condition, potentially without ever leaving the vessel.

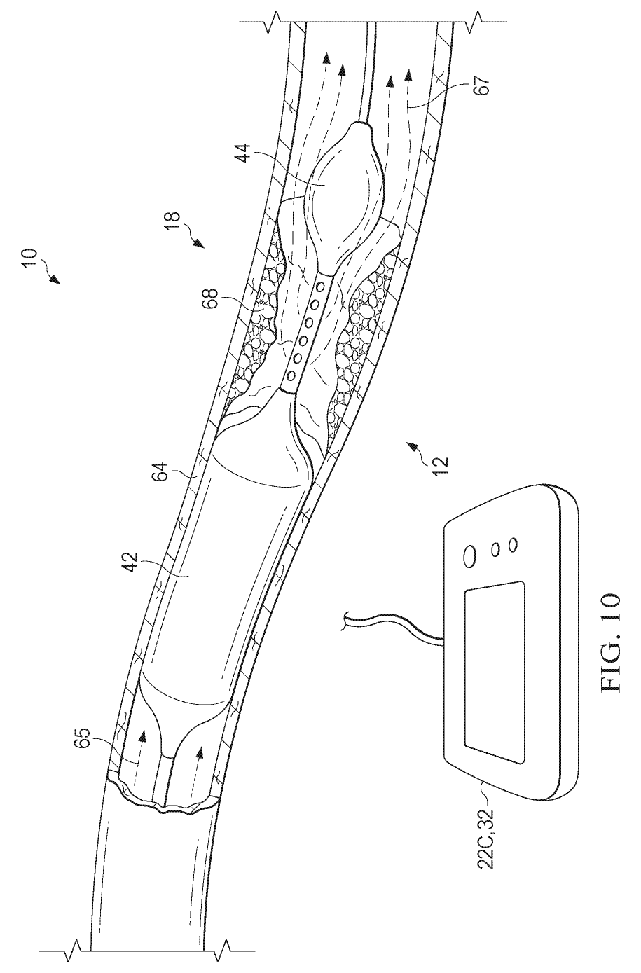

FIG. 10 through FIG. 12 illustrate the system 10 in exemplary use in an exemplary blood vessel 64. FIG. 10 illustrates the occluding balloon 42 inflated to substantially or fully block a normal flow of blood or other fluid 65 within the vessel 64 upstream of an area of study, such as with one or more occlusions 68. The occluding balloon 42 may be positioned upstream of, or at an upstream portion of, an area of study. The variable size balloon 44 may be positioned downstream of, or at a downstream portion of, the area of study. A flow 67 may be induced within the area of study, such as by releasing fluid through the ports 40 in the device 12.

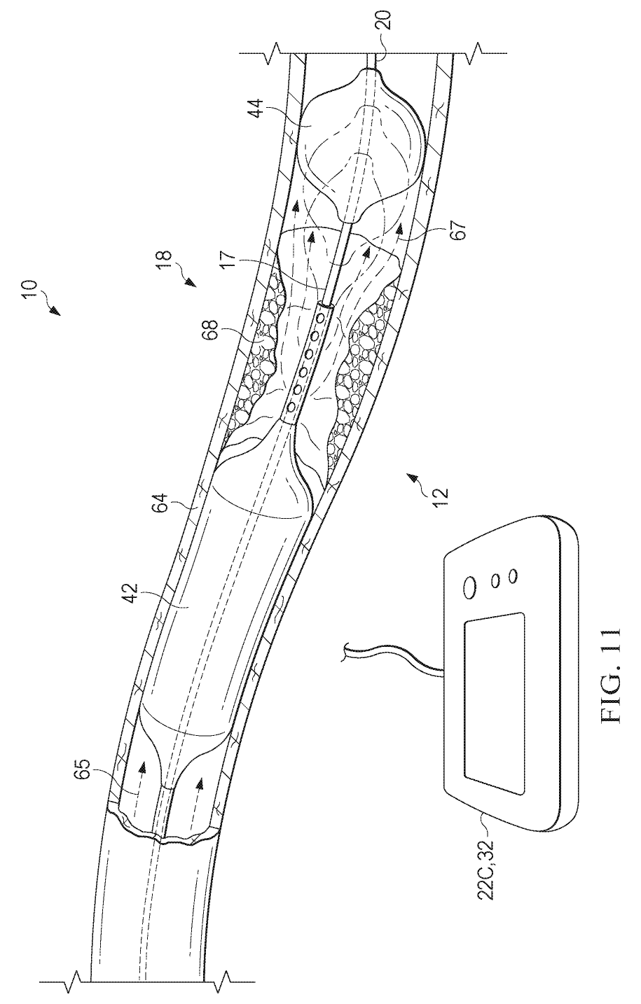

As illustrated in FIG. 11, the variable size balloon(s) 44 may be inflated to various sizes to measure changes to induced flow 67 resistance or other characteristics. Alternatively, or additionally, changes to the induced flow 67 may be implemented to measure changes in resistance or other characteristics. The distal balloon(s) 44 may be inflated to fully or partially occlude the vessel 64.

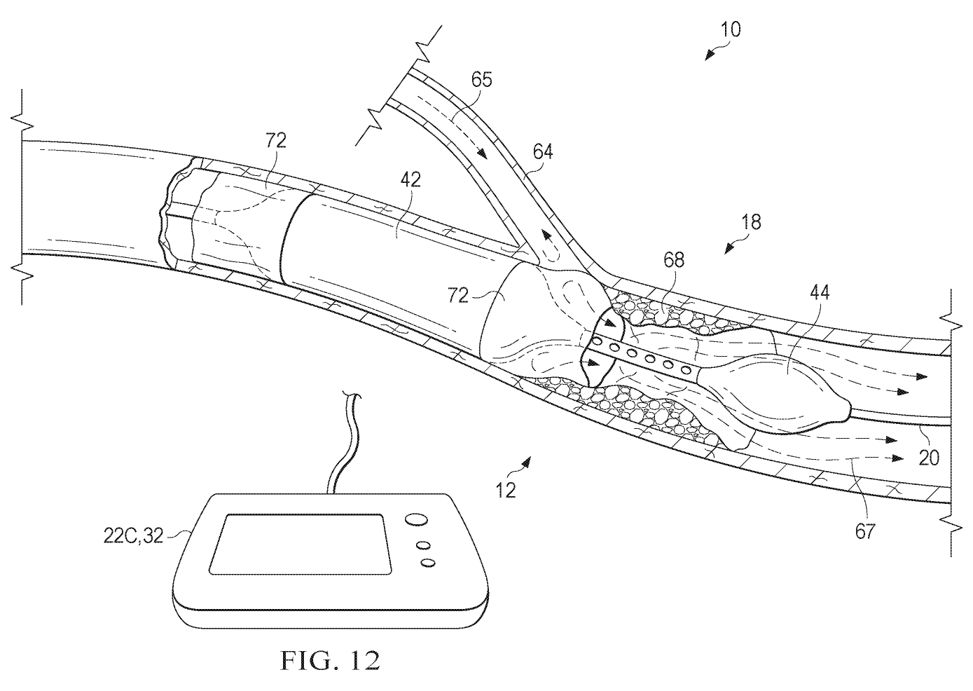

As illustrated in FIG. 12, the first balloon 42 may include one or more skirts 72. The skirts 72 may extend from one or both of the forward or rear portion of the first balloon 42. The skirts 72 may be moved by the first balloon 42 and/or natural forces of the natural flow 65 within the vessel 64 to be positioned against an interior wall of the vessel 64. This may further occlude the vessel 64, such as by ensuring a good seal against the interior wall of the vessel 64. Alternatively, or additionally, the skirt(s) 72 may fully or partially seal adjacent branch vessels 64, such as by blocking the flow 65 from those branches 64 from entering or exiting the area of study. This may be particularly useful where the area of study and/or occlusion 68 is proximate to one or more vessel 64 branches. The skirts 72 may, alternatively or additionally, smooth or block abnormalities in the vessel 64 not otherwise under study.

The skirt(s) 72 may allow the balloons 42, 44 to operate at relatively lower pressures by providing additional sealing and preventing natural flow 65 from entering the area of study.

While veins are sometimes discussed herein, these disclosures may be utilized for arterial other vasculature applications, by way of further non-limiting example.

Any embodiment of the present invention may include any of the features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention.

Certain operations described herein may be performed by one or more electronic devices. Each electronic device may comprise one or more processors, electronic storage devices, executable software instructions, combinations thereof, and the like configured to perform the operations described herein. The electronic devices may be general purpose computers or specialized computing devices. The electronic devices may comprise personal computers, smartphone, tablets, databases, servers, or the like. The electronic connections and transmissions described herein may be accomplished by wired or wireless means. The computerized hardware, software, components, systems, steps, methods, and/or processes described herein may serve to improve the speed of the computerized hardware, software, systems, steps, methods, and/or processes described herein. The electronic devices, including but not necessarily limited to the electronic storage devices, databases, controllers, or the like, may comprise and/or be configured to hold, solely non-transitory signals.

What is claimed is:

1. A system for interpreting vascular conditions of a blood vessel, said system comprising:
an intravascular device comprising:
a catheter tube;
a selectively inflatable first balloon located at a distal portion of said catheter tube;
a selectively inflatable second balloon located distal to, and spaced apart from, said first balloon along said catheter tube; and
one or more ports spaced apart axially along the catheter tube between the first balloon and the second balloon;
one or more sensors in fluid communication with the one or more ports and configured to measure one or more characteristics of fluids of the vascular system between the first balloon and the second balloon, said one or more characteristics comprising at least one of pressure and flow rate; and
a controller in electronic communication with the one or more sensors, wherein the controller comprises one or more processors and one or more non-transitory electronic storage devices storing software instructions, which when executed, cause the one or more processors to:
receive readings from the one or more sensors at instances of time;
determine a resistance to flow of the fluids for the instances of time based, at least in part, on the readings received from the one or more sensors; and
output a quantitative and/or qualitative indication of a vascular condition of the blood vessel based, at least in part, on at least one of: the resistance to flow at the respective one of the instances of time, and changes to the resistance to flow over at least some of the instances of time.

2. The system of claim 1, wherein a spacing of the first balloon relative to the second balloon is adjustable.

3. The system of claim 2, wherein the intravascular device further comprises an additional catheter tube, wherein the second balloon is fixed to the additional catheter tube, the first balloon is fixed to the catheter tube, and the additional catheter tube is moveable within the catheter tube in a sliding fashion to adjust the spacing of the first balloon relative to the second balloon.

4. The system of claim 2, wherein:
the instances of time include a first instance of time while a first bolus of the fluid is introduced from the one or more ports, and a second instance of time which is while or after at least one of: a second bolus of the fluid is introduced from the one or more ports, an inflation level of the second balloon is adjusted, and the spacing between the first balloon and the second balloon is adjusted; and
the indication of the vascular condition of the blood vessel is determined based, at least in part, on changes to the resistance to flow at the first instance of time and the second instance of time.

5. The system of claim 1, wherein the second balloon has a compliance level which is different from, and greater than, a compliance level of the first balloon.

6. The system of claim 5, wherein the second balloon is configured to fully or substantially occlude the blood vessel at a relatively lower inflation pressure than the first balloon.

7. The system of claim 1, wherein the first balloon is adjustably inflatable to a plurality of diameters to vary a clearance for fluid flow about the first balloon and a proximate section of the blood vessel.

8. The system of claim 1, wherein the one or more sensors are located at the intravascular device.

9. The system of claim 1, wherein the one or more sensors are located separately from the intravascular device and are fluidly connected thereto.

10. The system of claim 1, wherein the intravascular device further comprises:
a handle assembly;
a control device provided at the handle assembly which controls provision of a fluid from a fluid reservoir located at, or in fluid connection with, a proximal end portion of the catheter tube into a patient’s vascular system by way of the one or more ports to thereby become one of the fluids;
one or more additional control devices associated with the handle assembly which control provision of an inflation fluid from one or more additional fluid reservoirs located at, or in fluid connection with, a proximal end portion of the catheter tube into the first balloon and the second balloon such that the first balloon and the second balloon are separately inflatable to different inflation levels; and
one or more tubular passageways for the inflation fluid extending along or within the catheter tube and fluidly connected to the first balloon and the second balloon to facilitate inflation of the same.

11. The system of claim 1, wherein the intravascular device further comprises a skirt extending from at least one of a forward portion and a rear portion of the first balloon, said skirt configured to be positioned against an interior wall of the blood vessel to seal against the interior wall and to permit the first balloon and the second balloon to operate at relatively lower inflation pressures than if the skirt were not present.

12. The system of claim 1, further comprising one or more audio generating devices in electronic communication with the controller and configured to provide audible feedback indicative of the resistance to flow, wherein the audible feedback comprises a clicking sound and a pace of the clicking sound varies with the resistance to flow.

13. The system of claim 1, wherein the intravascular device further comprises a third selectively inflatable balloon located distal to said second balloon along said catheter tube, wherein the second balloon and the third balloon are individually inflatable to different inflation levels.

14. A system for interpreting vascular conditions of a blood vessel, said system comprising:
an intravascular device comprising:
a catheter tube;
a selectively inflatable first balloon located at a distal portion of said catheter tube, wherein the first balloon is adjustably inflatable to a plurality of diameters to vary a clearance for fluid flow about the first balloon and a proximate section of the blood vessel;
a selectively inflatable second balloon located distal to, and spaced apart from, said first balloon along said catheter tube, wherein a spacing of the first balloon relative to the second balloon is adjustable, wherein the second balloon has a compliance level which is different from, and greater than, a compliance level of the first balloon, and wherein the second balloon is configured to fully or substantially occlude the blood vessel at a relatively lower inflation pressure than the first balloon; and
one or more ports, open to the vascular system, spaced apart axially along the catheter tube between the first balloon and the second balloon;
one or more sensors in fluid communication with the one or more ports, and thereby the vascular system, and configured to measure one or more characteristics of fluids of the vascular system between the first balloon and the second balloon, said one or more characteristics comprising at least one of pressure and flow rate; and
a controller in electronic communication with the one or more sensors, wherein the controller comprises one or more processors and one or more non-transitory electronic storage devices storing software instructions, which when executed, cause the one or more processors to:
receive readings from the one or more sensors at instances of time;
determine a resistance to flow of the fluids for the instances of time based, at least in part, on the readings received from the one or more sensors; and
output a quantitative and/or qualitative indication of a vascular condition of the blood vessel based, at least in part, on at least one of: the resistance to flow at the respective one of the instances of time, and changes to the resistance to flow over at least some of the instances of time.

15. A method for interpreting vascular conditions of a blood vessel, said method comprising:
providing an intravascular device comprising:
a catheter tube;
a selectively inflatable first balloon located at a distal portion of said catheter tube;
a selectively inflatable second balloon located distal to, and spaced apart from, said first balloon along said catheter tube; and
one or more ports spaced apart axially along the catheter tube between the first balloon and the second balloon;
introducing the distal portion of the intravascular device into a patient’s vascular system;
inflating the first balloon to at least contact an interior surface of the blood vessel;
inflating the second balloon to at least partially occlude the blood vessel;
introducing a bolus of fluid into the patient’s vascular system from the one or more ports of the distal portion of the intravascular device;
measuring, by way of one or more sensors in fluid communication with the one or more ports, one or more characteristics of fluids of the vascular system between the first balloon and the second balloon at a plurality of instances of time, said one or more characteristics comprising at least one of pressure and flow rate;
determining a resistance to flow at the plurality of instances of time based, at least in part, on readings received from the one or more sensors at the plurality of instances of time; and
outputting a quantitative and/or qualitative indication of a vascular condition of the blood vessel based, at least in part, on at least one of: the resistance to flow at the respective one of the instances of time, and changes to the resistance to flow over the plurality of instances of time.

16. The method of claim 15 further comprising moving the second balloon relative to the first balloon to decrease a spacing between the first balloon and the second balloon, wherein the plurality of instances of time comprises at least one instance of time before the second balloon is moved relative to the first balloon and at least one instance of time after the second balloon is moved relative to the first balloon.

17. The method of claim 15 further comprising altering at least one of the one or more characteristics of a second bolus of the fluid relative to the bolus of the fluid in a known manner, wherein the plurality of instances of time comprises at least one instance of time before the second bolus of the fluid is introduced and at least one instance of time after the second bolus of the fluid is introduced.

18. The method of claim 15 further comprising adjusting an inflation level of the second balloon, wherein the plurality of instances of time comprises at least one instance of time before the inflation level of the second balloon is adjusted and at least one instance of time after the inflation level of the second balloon is adjusted.

19. The method of claim 15, further comprising:
performing, while the distal portion of the intravascular device remains within the patient’s vascular system, a therapy on the blood vessel using at least one of the first balloon and the second balloon, wherein the therapy comprises at least one of angioplasty, stenting, and drug delivery; and
measuring, by way of the one or more sensors after the therapy is performed and while the distal portion of the intravascular device remains within the patient’s vascular system, one or more further characteristics of the fluids of the vascular system between the first balloon and the second balloon to determine an effect of the therapy on the vascular condition of the blood vessel.

20. The method of claim 15, further comprising:
moving the distal portion of the intravascular device along a length of the blood vessel to at least a second position; and
at the second position, further measuring, by way of the one or more sensors, one or more further characteristics of the fluids of the vascular system between the first balloon and the second balloon at a plurality of further instances of time to determine a change in the vascular condition along the length of the blood vessel.
